(19) United States
(12) Patent Application Publication
(10) Pub. No.: US 2024/0101503 A1
(43) Pub. Date: Mar. 28, 2024

(54) Process for the Production of Cannabinoids and Cannabinoid Acids

(71) Applicant: Bessor Pharma, LLC, Framingham, MA (US)

(72) Inventors: Barry A. Berkowitz, Framingham, MA (US); Anthony G. Barrett, Leme, Rio de Janeiro (BR); Daniel Elliott, Basel (CH)

(21) Appl. No.: 17/767,760

(22) PCT Filed: Oct. 7, 2020
(86) PCT No.: PCT/US2020/054525

§ 371 Date: Apr. 8, 2022

Related U.S. Application Data
(60) Provisional application No. 62/912,131, filed on Oct. 8, 2019.

(51) Int. Cl. C07C 51/09; C07C 37/50; C07D 319/08

(52) U.S. Cl. CPC C07C 51/09; C07C 37/50; C07D 319/08

(57) ABSTRACT

The present invention relates to a process for the preparation of diverse known and novel cannabinoids 5, which include cannabigerol (CBG, 1), cannabigerolic acid (CBGA, 2), cannabigerovarin (CBGV, 3), cannabigerovarinic acid (CBGVA, 4) and other naturally occurring monocyclic cannabinoids and other analogues from simple inexpensive starting materials using a cascade sequence of allylic rearrangement and aromatization. Novel cannabinoids of series 5 are also claimed as part of the invention. These synthesized cannabinoids, unlike the minor cannabinoids isolated from Cannabis saliva or synthesized from the condensation reactions such as the reactions of substituted resorcinols with monoterpenes, are much easier to obtain at high purity levels. In particular, these cannabinoids, including but not limited to cannabigerol (CBG, 1), cannabigerolic acid (CBGA, 2), cannabigerovarin (CBGV, 3) and cannabigerovarinic acid (CBGVA, 4) are obtained without contamination with impurities with variation in RA and RB (e.g. contamination of CBG with CBGV).

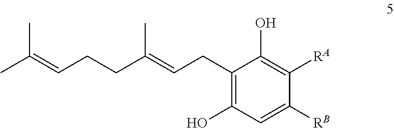

### FIELD OF THE INVENTION

The field of the invention relates to methods for the synthesis of high purity known and novel cannabinoids including but not limited to cannabigerol (CBG, 1), cannabigerolic acid (CBGA, 2), cannabigerovarin (CBGV, 3), cannabigerovarinic acid (CBGVA, 4) and other naturally occurring cannabinoids and other synthetic analogues from simple inexpensive starting materials by construction of the aromatic core. The field of the invention additionally covers novel cannabinoids, which may be used as active compounds either alone or admixed in combination with known cannabinoids or other drugs in drug formulations for the treatment of pain, multiple sclerosis-related spasticity, nausea, anorexia, epilepsy, Alzheimer's and other neurodegenerative diseases, brain injury/concussion/traumatic brain injury, stroke, cancer, infection, reduction of inflammation and immuno-inflammation related diseases, diseases/injury of the eye including but not limited to glaucoma, dry eye, corneal injury or disease and retinal degeneration or disease, disorders of immune-inflammation, lung injury or disease, liver injury or disease, kidney injury or disease, pancreatitis and disorders of the pancreas cardiovascular injury or disease, and organ transplant, reduction of post-surgical inflammation among other diseases, and as anti-oxidants.

### BACKGROUND OF THE INVENTION

Cannabis sativa (“marijuana”) is a hemp plant of considerable notoriety and use. Its use as a recreational drug worldwide, has been and remains the subject of legal review in many countries of the world. There has been very considerable interest in the use of this plant and its extracts as ethnopharmaceuticals for millennia with reference even in Herodotus, (The Histories, Book IV, page 295, Penguin Books, Ltd., Middlesex (1972). The plant and its extracts have been used in medicine on account of their effects as anesthetics, spasmolytics, and hypnotic agents, immune-inflammation regulatory agents to combat the side effects of nausea following cancer chemotherapy, in the treatment of glaucoma, neuropathic pain, epilepsy, multiple sclerosis-related spasticity and pain in patients with advanced cancer, AIDS-related anorexia and pain.

There are over 60 constituent compounds that have been isolated and characterized from Cannabis sativa oil (for example see S. A. Ahmed, S. A. Ross, D. Slade, M. M. Radwan, F. Zulfiqar and M. A. ElSohly “Cannabinoid Ester Constituents from High-Potency Cannabis sativa”, Journal of Natural Products, 2008, volume 71, pages 536-542; Lewis, M. M.; Yang, Y.; Wasilewski, E.; Clarke, H. A.; Kotra, L. P., “Chemical Profiling of Medical Cannabis Extracts”, ACS Omega, 2017, volume 2, pages 6091-6103 and references therein). In addition, a considerable number of these natural products and analogs have been prepared by total synthesis from aromatic and monoterpene precursor compounds. Such total syntheses are reported (for examples see R. K. Razdan, “The Total Synthesis of Cannabinoids” in “The Total Synthesis of Natural Products”, Editor J. ApSimon, 1996, volume 4, pages 185-262, New York, N.Y.: Wiley and Sons; J. W. Huffman and J. A. H. Lainton, “Recent Developments in the Medicinal Chemistry of Cannabinoids”, Current Medicinal Chemistry, 1996, volume 3, pages 101-116; N. Itagaki, T. Sugahara and Y. Iwabuchi, “Expedient Synthesis of Potent Cannabinoid Receptor Agonist (−)-CP55,940”, Organic Letters, 2005, volume 7, pages 4181-4183; J. A. Teske and A. Deiters, “A Cyclotrimerization Route to Cannabinoids”, Organic Letters, 2008, volume 10, pages 2195-2198; S. Tchilibon and R. Mechoulam, “Synthesis of a Primary Metabolite of Cannabidiol”, Organic Letters, 2000, volume 2, pages 3301-3303; Y. Song, S. Hwang, P. Gong, D. Kim and S. Kim*, “Stereoselective Total Synthesis of (−)-Perrottetinene and Assignment of Its Absolute Configuration”, Organic Letters, 2008, volume 10, pages 269-271; Y. Kobayashi, A. Takeuchi and Y.-G. Wang, “Synthesis of Cannabidiols via Alkenylation of Cyclohexenyl Monoacetate”, Organic Letters, 2006, volume 8, pages 2699-2702; B. M. Trost and K. Dogra, “Synthesis of (−)-Δ⁹-trans-Tetrahydrocannabinol: Stereocontrol via Mo-Catalyzed Asymmetric Allylic Alkylation Reaction”, Organic Letters, 2007, volume 9, pages 861-863; L.-J. Cheng, J.-H. Xie, Y. Chen, L.-X. Wang and Q.-L. Zhou, “Enantioselective Total Synthesis of (−)-Δ⁸-THC and (−)-Δ⁹-THC via Catalytic Asymmetric Hydrogenation and S_{N}Ar Cyclization” Organic Letters, 2013, volume 15, pages 764-767; P. R. Nandaluru and G. J. Bodwell, “Multicomponent Synthesis of 6H-Dibenzo[b,d]pyran-6-ones and a Total Synthesis of Cannabinol”, Organic Letters, 2012, volume 14, pages 310-313; S. Ben-Shabat, L. O. Hanus, G. Katzavian and R. Gallily, “New Cannabidiol Derivatives: Synthesis, Binding to Cannabinoid Receptor, and Evaluation of Their Antiinflammatory Activity”, Journal of Medicinal Chemistry, 2006, volume 49, pages 1113-1117; A. Mahadevan, C. Siegel, B. R. Martin, M. E. Abood, I. Beletskaya and R. K. Razdan, “Novel Cannabinol Probes for CB1 and CB2 Cannabinoid Receptors”, Journal of Medicinal Chemistry, 2000, volume 43, pages 3778-3785; S. P. Nikas, S. O. Alapafuja, I. Papanastasiou, C. A. Paronis, V. G. Shukla, D. P. Papahatjis, A. L. Bowman, A. Halikhedkar, X. Han and A. Makriyannis, “Novel 1′,1′-Chain Substituted Hexahydrocannabinols: 9β-Hydroxy-3-(1-hexyl-cyclobut-1-yl)-hexahydrocannabinol (AM2389) a Highly Potent Cannabinoid Receptor 1 (CB1) Agonist”, Journal of Medicinal Chemistry, 2010, volume 53, pages 6996-7010; Kavarana, M. J.; Peet, R. C., “Bioenzymatic Synthesis Of THC-V, CBY And CBN and their use as Therapeutic Agents”, US Patent Application, 2017/0283837 Al; Winnicki, R.; Donsky, M.; Sun, M.; Peet, R., “Apparatus and Methods for Biosynthetic Production of Cannabinoids’, U.S. Pat. No. 9,879,292 B2; Giorgi, P. D.; Liautard, V.; Pucheault, M.; Antoniotti, S. “Biomimetic Cannabinoid Synthesis Revisited: Batch and Flow All-Catalytic Synthesis of (±)-ortho-Tetrahydrocannabinols and Analogues from Natural Feedstocks”, European Journal of Organic Chemistry, 2018, pages 1307-1311; Morimoto, S.; Komatsu, K.; Taura, F.; Shoyama, Y., “Enzymological Evidence for Cannabichromenic Acid Biosynthesis”, Journal of Natural Products, 1997, volume 60, pages 854-857; Saimoto, H.; Yoshida, K.; Murakami, T.; Morimoto, M.; Sashiwa, H.; Shigemasa, Y., “Effect of Calcium Reagents on Aldol Reactions of Phenolic Enolates with Aldehydes in Alcohol”, The Journal of Organic Chemistry, 1996, volume 61, pages 6768-6769; Pollastro, F.; Caprioglio, D.; Marotta, P.; Moriello, A. S.; De Petrocellis, L.; Taglialatela-Scafati, O.; Appendino, G., “Iodine-Promoted Aromatization of p-Menthane-Type Phytocannabinoids”, Journal of Natural Products, 2018, volume 81, pages 630-633; Bastola, K. P.; Hazekamp, A.; Verpoorte, R., “Synthesis and Spectroscopic Characterization of Cannabinolic Acid”, Planta Medica, 2007, volume 73, pages 273-275).

In the last twenty years it has become apparent that the cannabinoids are in a renaissance for diverse biomedical uses. The pharmacology of the cannabinoids has been shown to be associated with a number of receptors and mechanisms including cannabinoids receptors, GPCR receptors, serotonin receptors, modulation of several voltage-gated channels (including Ca²⁺, Na⁺, and various type of K⁺ channels), ligand-gated ion channels (i.e., GABA, glycine and TRPV), Toll like receptors, opioid receptors, NMDA or excitatory amino acids receptors, catecholamine receptors, enzymes regulating endocannabinoids, and ion-transporting membranes proteins such as transient potential receptor class (TRP) channels (L. De Petrocellis, M. Nabissi, G. Santoni and A. Ligresti, “Actions and Regulation of Ionotropic Cannabinoid Receptors”, Advances in Pharmacology, 2017, volume 80, pages 249-289; P. Morales and P. H. Reggio, “An Update on Non-CB₁, Non-CB₂ Cannabinoid Related G-Protein-Coupled Receptors”, Cannabis Cannabinoid Research, 2017, volume 2, pages 265-273). Thus, it would be helpful to have a new medicament or medicaments that include one or more cannabinoids for treatment of afflictions known to be treatable by affecting or using these physiological mechanisms.

The pharmacology of the cannabinoids is directly or indirectly receptor-mediated for example, by two G protein-coupled receptors, named CB₁ and CB₂, which have 44% sequence homology in humans. The CB₁ sub-type is the most widely expressed G protein-coupled receptor in the brain in regions, for example, that control motor, emotional, cognitive, sensory responses, perception of pain, thermoregulation, as well as cardiovascular, gastrointestinal, and respiratory physiology. It is localized in the central (CNS) and peripheral nervous systems including the olfactory bulb, cortical areas, parts of the basal ganglia, thalamus, hypothalamus, cerebellar cortex, brainstem, and spinal cord. CB₁ receptors also occur in cells in the pituitary and thyroid glands, some fat, muscle and liver cells as well as the lung and kidneys. The CB₂ sub-type is expressed in immune and hematopoietic cells, osteoclasts, and osteoblasts and mediates the response of the immune system, controls inflammation, modulates inflammatory and neuropathic pain as well as bone remodeling.

The pharmacology of modulators of CB₁ and CB₂ receptors has been reviewed for example by Vemuri and Makriyannis (V. K. Vemuri and A. Makriyannis, “Medicinal Chemistry of Cannabinoids”, Clinical Pharmacology & Therapeutics, 2015, volume 97, pages 553-558). The psychoactive effects of Δ⁹-tetrahydrocannabinol (THC) as well as with its primary metabolite 11-hydroxy-Δ⁹-tetrahydrocannabinol are mediated by its partial agonism of CNS CB₁ receptors (J. van Amsterdam, T. Brunt and W. van den Brink, “The adverse health effects of synthetic cannabinoids with emphasis on psychosis-like effects”, Journal of Psychopharmacology, 2015, volume 29, pages 254-263; R. G. Pertwee, “The diverse CB₁ and CB₂ receptor pharmacology of three plant cannabinoids: Δ⁹-tetrahydrocannabinol, cannabidiol and Δ⁹-tetrahydrocannabivarin”, British Journal of Pharmacology, 2008, volume 153, pages 199-215). It is useful as an analgesic, an antiemetic agent, and for treating anorexia in patients with AIDS. Other CB₁ receptor modulators include tetrahydrocannabivarin (THCV) (weak antagonist) and cannabinol (CBN) (weak agonist) and both are modest agonists of CB₂. Both the non-psychoactive (−)-cannabidiol (CBD) and cannabidivarin (CBDV) do not interact significantly with either receptor sub-class and their modes of action are less clear (J. Fernández-Ruiz, O. Sagredo, M. R. Pazos, C. Garcia, R. Pertwee, R. Mechoulam, J. Martinez-Orgado, “Cannabidiol for neurodegenerative disorders: important new clinical applications for this phytocannabinoid?”, British Journal of Clinical Pharmacology, 2013, volume 75, pages 323-333; S. Rosenthaler, B. Pöhn, C. Kolmanz, C. N. Huu, C. Krewenka, A. Huber, B. Kranner, W.-D. Rausch and R. Moldzio, “Differences in receptor binding affinity of several phytocannabinoids do not explain their effects on neural cell cultures”, Neurotoxicology and Teratology, 2014, volume 46, pages 49-56). The combination of Δ⁹-tetrahydrocannabinol (THC) and cannabidiol (CBD) (Sativex, Nabiximols) is used to treat multiple sclerosis-related spasticity and as a potent analgesic in patients with advanced stage cancers. More recently, purified cannabidiol (CBD) was granted orphan drug status for treating epilepsy. CB₁ receptor antagonists are appetite suppressants, enhance cognition, and control addictive behavior. Selective CB₂ agonists may provide superior analgesic agents and immunomodulators that do not have the undesirable psychoactive effects associated with CNS CB₁ agonism. Δ⁹-tetrahydrocannabinol (THC) (Dronabinol) has been shown to be clinically effective either in monotherapy or in combination with ondansetron (Zofran, a 5-HT₃ antagonists) and in combination with prochlorperazine (a dopamine D₂ receptor antagonist) to treat chemotherapy-induced nausea and vomiting in cancer patients (M. B. May and A. E Glode, “Dronabinol for chemotherapy-induced nausea and vomiting unresponsive to antiemetics”, Cancer Management and Research, 2016, volume 8, pages 49-55).

Cannabinoids that are used as therapeutics are either obtained from the fractionation of Cannabis sativa oil or from total synthesis usually from aromatic and terpene starting materials. Since there are over 60 different natural products in Cannabis oil, such oil fractionation requires extensive chromatographic purification to provide any individual constituent substantially pure (>99% pure) and, with so many components, makes reproducible production and storage difficult. For example, the purification of Δ⁹-tetrahydrocannabinol (THC) from other Cannabis constituents but particularly from its isomer Δ⁸-tetrahydrocannabinol is inefficient and costly. In addition, since many of the cannabinoids in Cannabis oil have different effects as total, partial, inverse or neutral agonists or antagonists of either or both of the CB₁ and CB₂ receptors, it is especially important that individual isolated natural products do not contain significant levels (below parts per million levels) of any other cannabinoid natural product, which has undesired biological effects and that the specifications set are efficiently reproducible. There is an added complication in that many cannabinoid natural products are obtained as oils, which are typically not possible to crystallize and which are prone to air oxidative degradation and their isolation requires the use of extensive expensive and difficult to scale chromatography and/or derivatisation (for example see B. Trawick and M. H. Owens, “Process for the Preparation of (−)-delta 9-Tetrahydrocannabinol”, WO 2009/099868 Al; E. Arslantas and U. Weigl, “Method for Obtaining Pure Tetrahydrocannabinol”, U.S. Pat. No. 7,923,558 B2; J. E. Field, J. Oudenes, B. I. Gorin, R. Orprecio, F. E. Silva e Souza, N.J. Ramjit and E.-L. Moore, “Separation of Tetrahydrocannabinols”, U.S. Pat. No. 7,321,047 B2; P. Bhatarah, K. J. Batchelor, D. McHattie and A. K. Greenwood, “Delta 9 Tetrahydrocannabinol Derivatives”, WO 2008/099183 Al; D. C. Burdick, S. J. Collier, F. Jos, B. Biolatto, B. J. Paul, H. Meckler, M. A. Helle and A. J. Habershaw, “Process for Production of Delta-9-Tetrahydrocannabinol”, U.S. Pat. No. 7,674,922 B2).

The cannabinoids cannabigerol (CBG, 1), cannabigerolic acid (CBGA, 2), cannabigerovarin (CBGV, 3), cannabigerovarinic acid (CBGVA, 4), have also been isolated and characterized from Cannabis sativa oil in variable purities. Cannabigerol (CBG, 1) is the second major phytocannabinoid in the Cannabis plant.

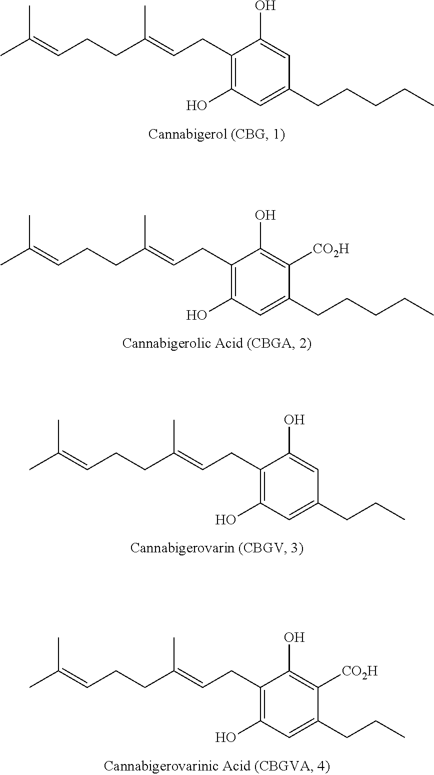

Many of the known synthetic routes to prepare cannabinoids either use expensive reagent and are uneconomic to use on a large scale or are dependent on the condensation reactions of monoterpene starting materials with derivatives of alkyl-resorcinol such as 5-n-pentyl-resorcinol (olivetol) under acidic reaction conditions, reactions that frequently give rise to side products derived from carbenium ion rearrangement reactions and/or side reactions. For example, the manufacture of Δ⁹-tetrahydrocannabinol (THC) from olivetol and monoterpenes by Brønsted or Lewis acid catalyzed condensation reactions is complicated by the co-formation of its isomer Δ⁸-tetrahydrocannabinol, amongst other impurities. Such impurities also considerably complicate and increase the cost of obtaining cannabinoid active pharmaceutical ingredients substantially pure (for examples see R. K. Razdan, “The Total Synthesis of Cannabinoids” in “The Total Synthesis of Natural Products”, Editor J. ApSimon, 1996, volume 4, pages 185-262, New York, N.Y.: Wiley and Sons; C. Steup and T. Herkenroth, “Process for Preparing Synthetic Cannabinoids”, US Patent Application 2010/0298579 A1; R. J. Kupper, “Cannabinoid Active Pharmaceutical Ingredient for Improved Dosage Forms”, WO 2006/133941 A2; J. Erler, and S. Heitner, “Method for the Preparation of Dronabinol”, U.S. Pat. No. 8,324,408 B2; A. L. Gutman, M. Etinger, I. Fedotev, R. Khanolkar, G. A. Nisnevich, B. Pertsikov, I. Rukhman and B. Tishin, “Methods for Purifying trans-(−)-Δ⁹-Tetrahydrocannabinol and trans-(+)-Δ⁹-Tetrahydrocannabinol”, U.S. Pat. No. 9,278,083 B2).

Cannabigerol (1) has previously been synthesized from olivetol and geraniol by Lewis acid or Brønsted acid catalyzed condensation (S-H. Baek, C. N. Yook, D. S. Han, “Boron trifluoride etherate on alumina—a modified Lewis acid reagent(V) a convenient single-step synthesis of cannabinoids”, Bulletin of the Korean Chemical Society, 1995, volume 16, pages 293-6). In a similar fashion, cannabigerovarin (3) has been synthesized from 5-propyl resorcinol (M J. Kavarana, R. C. Peet, “Bioenzymatic Synthesis of THC-v, CBV and CBN and Their Use as Therapeutic Agents”, US20170283837 A1).

The syntheses of cannabigerolic acid (2) and cannabigerovarinic acid (4) have been carried out by reaction of cannabigerol (1) and cannabigerovarin (3), respectively, with magnesium methyl carbonate (R. Peet, M. Sun, “Apparatus and methods for the simultaneous production of compounds”, US 2016/0053220 A1).

Cannabigerol (CBG, 1) is non-psychotropic and has a low affinity for the CB1 receptor but inhibits the uptake of anandamide. It acts as a potent agonist of the a 2 adrenoceptor in mouse brain membranes. It additionally modulates 5HT1A receptors and, like many phytocannabinoids, cannabigerol (CBG, 1) modulates numerous TRP cation channels. It is a potent TRPA1 agonist, a weak agonist at TRPV1 and TRPV2 and a potent TRPM8 antagonist. It has been shown to have anti-cancer activity possibly via TRPM8 receptor antagonism and calcium signaling regulation. CBG (1) has been shown to be potentially useful for GI-GU disease including inflammatory bowel disease, colitis and in bladder control. CNS utility for CBG (1) has also been indicated based on action for models of neuro-inflammation, Huntington's disease, Parkinson's disease and encephalomyelitis including design and study of CBG derivatives and CBG (1) itself. It has been claimed to show appetite stimulation properties, that it is a regulator of immuno-inflammation and to have anti-oxidation properties (Turner, S. E.; Williams, C. M.; Iversen, L.; Whalley, B. J., “Molecular Pharmacology of Phytocannabinoids”, Phytocannabinoids, 2017, pages 61-101; Lewis, M. M.; Yang, Y.; Wasilewski, E.; Clarke, H. A.; Kotra, L. P., “Chemical Profiling of Medical Cannabis Extracts”, ACS Omega, 2017, volume 2, pages 6091-6103; Borrelli F, Pagano E, Romano B, Panzera S, Maiello F, Coppola D, De Petrocellis L, Buono L, Orlando P, Izzo AA, “Colon carcinogenesis is inhibited by the TRPM8 antagonist cannabigerol, a Cannabis-derived non-psychotropic cannabinoid” Carcinogenesis. 2014 December; 35(12):2787-97; Valdeolivas, S.; Navarrete, C.; Cantarero, I.; Bellido, M. L.; Muñoz, E.; Sagredo, O., “Neuroprotective properties of Cannabigerol in Huntington's disease: Studies in R6/2 Mice and 3-Nitropropionate-lesioned Mice”, Neurotherapeutics, 2015, volume 12, pages 185-99; Giacoppo, S.; Gugliandolo, A.; Trubiani, O.; Pollastro, F.; Grassi, G.; Bramanti, P.; Mazzon, E., “Cannabinoid CB2 receptors are involved in the protection of RAW264.7 macrophages against the oxidative stress: an in vitro study”, European Journal of Histochemistry, 2017, volume 61, page 2749; Gugliandolo, A.; Pollastro, F.; Grassi, G.; Bramanti, P.; Mazzon, E., “In Vitro Model of Neuroinflammation: Efficacy of Cannabigerol, a Non-Psychoactive Cannabinoid” International Journal of Molecular Sciences, 2018, volume 19, page 1992; Couch, D. G.; Maudslay, H.; Doleman, B.; Lund.; J. N.; O'Sullivan, S. E., “The Use of Cannabinoids in Colitis: A Systematic Review and Meta-Analysis”, Inflammatory Bowel Diseases, 2018, volume 24, pages 680-697; Garcia, C.; Gómez-Cañas, M.; Burgaz, S.; Palomares, B.; Gómez-Gálvez, Y.; Palomo-Garo, C.; Campo, S.; Ferrer-Hernandez, J.; Pavicic, C.; Navarrete, C.; Bellido, M. L.; Garcia-Arencibia, M.; Pazos, M. R.; Muñoz, E.; Fernandez-Ruiz, J., “Benefits of VCE-003.2, a cannabigerol quinone derivative, against inflammation-driven neuronal deterioration in experimental Parkinson's disease: possible involvement of different binding sites at the PPARγ receptor. Journal of Neuroinflammation”, 2018, volume 15, page, 19; Brierley, D. I.; Samuels, J.; Duncan, M.; Whalley, B. J.; Williams, C. M. “Cannabigerol is a novel, well-tolerated appetite stimulant in pre-satiated rats”, Psychopharmacology (Heidelberg), 2016, volume 233, pages 3603-13; Carrillo-Salinas, F. J.; Navarrete, C.; Mecha, M.; Feliú, A.; Collado, J. A.; Cantarero, I.; Bellido, M. L.; Muñoz, E.; Guaza, C., “A cannabigerol derivative suppresses immune responses and protects mice from experimental autoimmune encephalomyelitis”, PLoS One, 2014, volume 9, pages e94733; Pagano. E.; Montanaro, V.; Di Girolamo, A.; Pistone, A.; Altieri, V.; Zjawiony, J. K.; Izzo, A. A.; Capasso, R., “Effect of Non-psychotropic Plant-derived Cannabinoids on Bladder Contractility: Focus on Cannabigerol”, Natural Product Communications, 2015, volume 10, pages 1009-12.

The bioactivities of cannabigerovarin (CBGV, 3) have not been well studied. It has potential in the treatment of dry-skin syndrome and reduced arachidonic acid (AA)-induced ‘acne-like’ lipogenesis and as an anti-inflammatory agent. Additionally, it acts at TRP cation channels for example as an agonist on TRPA1 and it desensitizes other TRP channels (for example see Shoyama, Y.; Hirano, H.; Oda, M.; Somehara, T.; Nishioka, I., Cannabis IX Cannabichromevarin and cannabigerovarin, two new propyl homologs of cannabichromene and cannabigerol”, Chemical & Pharmaceutical Bulletin, 1975, volume 23, pages 1894-1895; De Petrocellis, L.; Orlando, P.; Moriello, A. S.; Aviello, G.; Stott, C.; Izzo, A. A.; Di Marzo, V., Cannabinoid actions at TRPV channels: effects on TRPV3 and TRPV4 and their potential relevance to gastrointestinal inflammation”, Acta Physiologica, 2012, volume 204, pages 255-266; Petrosino, S.; Verde, R.; Vaia, M.; Allarà, M.; Iuvone, T.; Di Marzo, V., “Anti-inflammatory Properties of Cannabidiol, a Nonpsychotropic Cannabinoid, in Experimental Allergic Contact Dermatitis”, Journal of Pharmacology and Experimental Therapeutics, 2018, volume 365, pages 652-663.

The cannabinoid carboxylic acids cannabigerolic acid (CBGA, 2) and cannabigerovarinic acid (CBGVA, 4), currently have limited biological and medical applications. Cannabigerolic acid (CBGA, 2) has been claimed to be a modest modulator of the inhibition of ovarian, breast, lung, pancreas and other cancer cell growth by cannabidiol (CBD) and cannabigerol (CBG, 1) and itself to kill breast cancer cells. It is an inverse agonist of the G-protein Coupled Receptor GPR55, an antagonist of mono-acyl-glyceride lipase and a dual PPARα/γ agonist. Cannabigerolic acid (CBGA, 2) has also been suggested to show analgesic effects. Cannabigerovarinic acid (CBGVA, 4) is reported to have anticancer cytostatic effects at high doses in vitro on leukemia cells. It has been claimed that mixtures of CBGA (2), CBGVA (4) or another cannabinoid with mitragynine, pseudoindoxyl or 7-hydroxymitragynine and another additive may be used to treat inflammation, spasms or pain. Based alone on an in vitro cellular assay, cannabigerolic acid (CBGA, 2), amongst other acidic cannabinoids, have been claimed to be of use in increasing the natural resistance of an animal, enhancing cellular resistance, for treating diabetes or atherosclerosis and in reducing the decline in stress response found in ageing. (D'Aniello, E.; Fellous, T.; Iannotti, F. A.; Gentile, A.; Allarà, M.; Balestrieri, F.; Gray, R.; Amodeo, P.; Vitale, R. M.; Di Marzo, V., “Identification and characterization of phytocannabinoids as novel dual PPARα/γ agonists by a computational and in vitro experimental approach”, Biochimica et Biophysica Acta General Subjects, 2019, volume 183, pages 586-597; Korthout, H. A. A. J.; Verhoeckx, K. C. M.; Witkamp, R. F.; Doornbos, R. P.; Mei Wang, M., “Medicinal Acidic Cannabinoids:, U.S. Pat. No. 7,807,711; Parolaro, D.; Massi, P., Antonio, A.; Francesca Borelli, F.; Aviello, G.; Di Marzo, V.; De Petrocellis, L.; Schiano Moriello, A. S.; Ligresti, A.; Alexandra Ross, R. A.; Ford, L. A.; Anavi-Goffer, S.; Guzman, M.; Velasco, G.; Lorente, M.; Torres, S.; Kikuchi, T.; Guy, G.; Stott, C.; Wright, S.; Sutton, A.; Potter, D.; Etienne De Meijer, E., “Phytocannabinoids in the Treatment of Cancer”, U.S. Pat. No. 8,790,719; Javid, F. A.; Duncan, M.; Stott, C., “Use of Phytocannabinoids in the Treatment of Ovarian Carcinoma”, U.S. Pat. No. 10,098,867; Stott, C.; Duncan, M.; Hill, T., “Active Pharmaceutical Ingredient (API) Comprising Cannabinoids for use in the Treatment of Cancer”, U.S. Pat. No. 9,962,341; Scott, K. A.; Shah, S.; Dalgleish, A. G.; Liu, W. M., “Enhancing the Activity of Cannabidiol and Other Cannabinoids In Vitro Through Modifications to Drug Combinations and Treatment Schedules”, Anticancer Research, 2013, volume 33, pages 4373-4380; Ahmed, S. A.; Ross, S. A.; Slade, D.; Radwan, M. M.; Zulfiqar, F.; ElSohly, M. A., “Cannabinoid Ester Constituents from High-Potency Cannabis sativa”, Journal of Natural Products, 2008 volume 71, pages 536-542; Kariman, A., “Compound and Method for Treating Spasms, Inflammation and Pain”, US Patent Application US 2018/0193399 A1; Korthout, H. A. A. J., “Medical use for Acidic Cannabinoids”, WO Patent Application 2012/144892 A1; Wright, S.; Wilhu, J., Parenteral formulations”, GB Application 2551986).

A vast number of combinations of one, two or three cannabinoids including cannabigerolic acid (CBGA, 2), cannabigerovarinic acid (CBGVA, 4) admixed with terpenes have been claimed but their possible uses have not been defined (Levy, K.; Cooper, J. M.; Martin, J. R.; Reid, B. G., “Compositions Purposefully Selected Comprising Purified Cannabinoids and/or Purified Terpenes”, WO Patent Application 2018/160827 A1).

In contrast to these currently limited biomedical applications for the cannabinoid acids 2 and 4, THCA, which is the carboxylic acid precursor of THC, has been widely studied. In a series of preclinical studies, THCA has been shown to be of value in controlling pain including neuropathic pain and fibromyalgia, epilepsy, cancers of the prostate, breast, colon, lung and skin, inflammation including encephalomyelitis as well as autoimmune diseases and to act as an anti-emetic (for examples see Dejana, R. Z.; Folić, M.; Tantoush, Z.; Radovanović, M.; Babić, G.; Janković, S. M., “Investigational cannabinoids in seizure disorders, what have we learned thus far?” Expert Opinion on Investigational Drugs, 2018, volume 27, pages 535-541; Rock, E. M.; Kopstick, L.; Limebeer, C. L.; Parker, L. A., “Tetrahydrocannabinolic acid reduces nausea-induced conditioned gaping in rats and vomiting in Suncus murinus”, British Journal of Pharmacology, 2013, volume 170, pages 641-648; Korthout, H. A. A. J; Verhoeckx, K. C. M.; Witkamp, R. F.; Doornbos, R. P.; Wang, M., “Medicinal Acidic Cannabinoids”, U.S. Pat. No. 7,807,711 B2; Rock, E. M.; Limebeer, C. L.; Navaratnam, R.; Sticht, M. A.; Bonner, N.; Engeland, K.; Downey, R.; Morris, H.; Jackson, M.; Parker, L. A., “A comparison of cannabidiolic acid with other treatments for anticipatory nausea using a rat model of contextually elicited conditioned gaping”, Psychopharmacology, 2014, volume 231, pages 3207-3215; Di Marzo, V.; De Petrocellis, L.; Moriello, A. S., “New use for cannabinoid-containing plant extracts”, G. B. Patent 2,448,535; Parolaro, D.; Massi, P.; Izzo, A. A.; Borelli, F.; Aviello, G.; Di Marzo, V.; De Petrocellis, L.; Moriello, A. S.; Ligresti, A.; Ross, R. A.; Ford, L. A.; Anavi-Goffer, S.; Guzman, M.; Velasco, G.; Lorente, M.; Torres, S.; Kikuchi, T.; Guy, G.; Stott, C.; Wright, S.; Sutton, A.; Potter, D.; De Meijer, E., “Phytocannabinoids in the Treatment of Cancer”, U.S. Pat. No. 8,790,719 B2; Trevor Percival Castor, T. P.; Rosenberry, L. C.; Tyler, T. A.; Student, R. J., “Methods for Making Compositions and Compositions for Treating Pain and Cachexia”, US Patent Application 2008/0103193 Al; Kariman, K., “Compound and Method for Treating Spasms, Inflammation and Pain”, US Patent Application 2018/0193399 Al; Sinai, A.; Turner, Z., “Use of Cannabis to Treat Fibromyalgia, Methods and Compositions Thereof”, WO Patent Application 2016/181394 Al).

If the cannabinoid acids 2 and 4 were to be made available more easily in larger quantities and higher purities, it would be possible to better and more thoroughly examine their uses in medicine either as mono-therapeutic agents or in combination with other cannabinoids or other biologically active compounds. It is germane to note that mixtures of cannabinoids may be more efficacious than single components (the entourage effect). For example, the presence of THCA and other cannabinoids has been shown to enhance the efficacy of THC as an antitumor agent in cell culture and animal models of ER+/PR+, HER2+ and triple-negative breast cancer (for example see Blasco-Benito, S.; Seijo-Vila, M.; Caro-Villalobosa, M.; Tundidor, I.; Andradas, C.; Garcia-Taboada, E.; Wade, J.; Smith, S.; Guzmán, M.; Perez-Gómez, E.; Gordon, M.; Sánchez, C., “Appraising the “entourage effect”: Antitumor action of a pure cannabinoid versus a botanical drug preparation in preclinical models of breast cancer”, Biochemical Pharmacology, 2018, volume 157, pages 285-293).

The present invention is directed towards overcoming the problems of availability of all the cannabinoids 1 to 4 in high purities by providing efficient/reproducible manufacturing routes for these compounds and providing flexible syntheses of novel cannabinoid analogs, which may be used as active compounds either alone or admixed in combination with known cannabinoids or other drugs in drug formulations for the treatment of pain, multiple sclerosis-related spasticity, nausea, anorexia, epilepsy, Alzheimer's and neurodegenerative diseases, brain injury/concussion/traumatic brain injury, stroke, cancer, infection, reduction of inflammation and immuno-inflammation related diseases, diseases/injury of the eye including but not limited to glaucoma, dry eye, corneal injury or disease and retinal degeneration or disease, disorders of immune-inflammation, lung injury or disease, liver injury or disease, kidney injury or disease, pancreatitis and disorders of the pancreas cardiovascular injury or disease, and organ transplant, reduction of post-surgical inflammation among other diseases, and as anti-oxidants.

### SUMMARY OF THE INVENTION

Among the benefits and improvements disclosed herein, other objects and advantages of the disclosed embodiments will become apparent from the following wherein like numerals represent like parts throughout the several figures. Detailed embodiments of cannabinoid compounds, intermediary compounds, and a process for preparation of cannabinoid and cannabimimetic compounds and their intermediaries are disclosed; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “In some embodiments” and “in some embodiments” as used herein do not necessarily refer to the same embodiment(s), though it may. The phrase “in another embodiment” as used herein does not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.

Further, the terms “substantial,” “substantially,” “similar,” “similarly,” “analogous,” “analogously,” “approximate,” “approximately,” and any combination thereof mean that differences between compared features or characteristics is less than 25% of the respective values/magnitudes in which the compared features or characteristics are measured and/or defined.

The purpose of combination or adjuvant therapies herein described are to enhance the efficacy of a drug by the use of a second drug or more drugs or to reduce the dose-limiting toxicities of a drug by the use of a second drug or more drugs.

As used herein, the term “substituted benzyl” means a benzyl ring bearing 1, 2 or 3 independently varied C1-C4 alkyl, C1-C4 alkyloxy, fluoro, chloro, hydroxy, trifluoromethyl, trifluoromethoxy, methylenedioxy, cyano, or methoxymethyl groups at an aromatic ring position or positions or 1 or 2 independently varied C1-C4 alkyl at the benzylic methylene.

If not otherwise defined herein, the term “optionally substituted aryl” means a phenyl ring optionally bearing 1, 2, or 3 independently varied C1-C4 alkyl, C1-C4 alkyloxy, fluoro, or chloro groups.

If not otherwise defined herein, the term “substituted” means optionally substituted at any position with varied C1-C4 alkyl, C1-C4 alkyloxy, fluoro, chloro, hydroxy, trifluoromethyl, trifluoromethoxy, methylenedioxy, cyano, or methoxymethyl groups.

The present invention relates to a process for the preparation of diverse known and novel cannabinoids 5 from the precursors 6 via the intermediates 7 including cannabigerol (CBG, 1), cannabigerolic acid (CBGA, 2), cannabigerovarin (CBGV, 3) and cannabigerovarinic acid (CBGVA, 4) and other naturally occurring monocyclic cannabinoids and other synthetic monocyclic analogues from simple inexpensive starting materials using a cascade sequence of allylic rearrangement and aromatization.

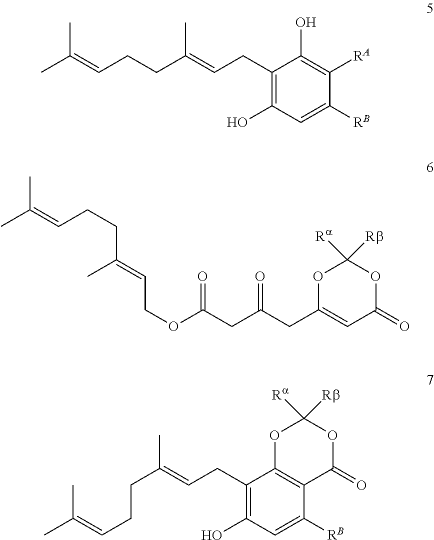

wherein:
R^{A} is H, CO₂H and its pharmaceutically acceptable salts, CO₂R^{C}, CONHR^{D}, CONR^{D}R^{E}.
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl, (CH₂)ᵣ—C₃ to C₆ cycloalkyl;
each r is independently 0, 1, 2, 3, 4, 5 or 6;
Rα and Rβ are independently C₁ to C₆ alkyl or optionally substituted aryl or Rα and Rβ in combination are (CH₂)ₛ (s is 4, 5 or 6) with Rα and Rβ being preferably both methyl.

The synthetic methods are suitable for use on a large scale and for manufacturing purposes. Examples of known cannabinoids that are available using the synthetic routes are cannabigerol (CBG, 1), cannabigerolic acid (CBGA, 2), cannabigerovarin (CBGV, 3) and cannabigerovarinic acid (CBGVA, 4). The synthetic methods are also suitable for the synthesis of novel cannabinoids and these compounds are also part of the invention. The cannabinoids 5 below, which are novel analogs of cannabigerol (CBG, 1), cannabigerolic acid (CBGA, 2), cannabigerovarin (CBGV, 3) and cannabigerovarinic acid (CBGVA, 4), are also available by the synthetic routes herein described and are part of the invention. These cannabinoids 5 have the formula:

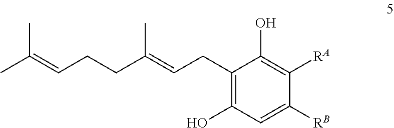

wherein:
R^{A} is H, CO₂H and its pharmaceutically acceptable salts, CO₂R^{C}, CONHR^{D}, CONR^{D}R^{E};
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl with the exclusion of R^{B} being n-propyl or n-pentyl, when R^{A} is H or CO₂H;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl, (CH₂)ᵣ—C₃ to C₆ cycloalkyl;
each r is independently 0, 1, 2, 3, 4, 5 or 6.

The aforementioned novel cannabinoids with the limited formulae 1-4 above may be used as active compounds either alone or admixed in combination with known cannabinoids such as but not limited to Δ⁹-tetrahydrocannabinol (THC), tetrahydrocannabivarin (THCV), cannabidiol (CBD) or cannabidivarin (CBVD) alone or in combination or with other drugs for the treatment of pain, multiple sclerosis-related spasticity, nausea, epilepsy, Alzheimer's brain injury/concussion, cancer, infection, glaucoma and retinal degeneration, disorders of immune-inflammation, lung injury or disease, liver injury or disease, kidney injury or disease, eye injury or disease, amongst other pathologies. In some embodiments, the said novel cannabinoids with the limited formulae 5 above either alone or admixed in combination with known cannabinoids such as but not limited to Δ⁹-tetrahydrocannabinol (THC), tetrahydrocannabivarin (THCV), cannabidiol (CBD), or cannabidivarin (CBDV) alone or in combination or with other drugs are formulated into pharmaceutical compositions in a suitable form for administration to a patient. Such formulations, in addition to the active cannabinoid or cannabinoids or other drugs in a combination therapeutic agent, contain pharmaceutically acceptable diluents and excipients. In the context of this invention, the term excipient encompasses standard excipients well known to a person of ordinary skill in the art (for example see Niazi, S. K., “Handbook of Pharmaceutical Manufacturing Formulations, Compressed Solid Products, 2009, volume 1, pages 67 and 99-169 2^{nd} Edition, Informa Healthcare) but also may include a volatile or mixture of volatile synthetic or isolated monoterpenes from Cannabis sativa and citrus oil. The aforementioned pharmaceutical compositions may be administrated to a patient by enteral, sublingual, intranasal, inhalation, rectal or parenteral drug administration or by other known methods of clinical administration.

### DETAILED DESCRIPTION OF THE INVENTION

Large Scale-Synthesis of Cannabigerol (CBG, 1), Cannabigerolic Acid (CBGA, 2), Cannabigerovarin (CBGV, 3), Cannabigerovarinic Acid (CBGVA, 4) and Analogs

The present invention relates to a large-scale process for the preparation of diverse known and novel cannabinoids 5 including cannabigerol (CBG, 1), cannabigerolic acid (CBGA, 2), cannabigerovarin (CBGV, 3) and cannabigerovarinic acid (CBGVA, 4) and other naturally occurring monocyclic cannabinoids from simple inexpensive starting materials using a cascade sequence of allylic rearrangement and aromatization. The invention includes synthesis of the target cannabinoids as oils or crystalline derivatives, as appropriate, including solvates, hydrates and polymorphs. The process involves the large-scale syntheses of cannabinoids 5:

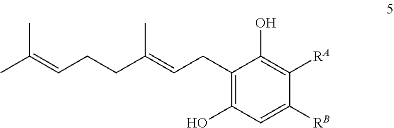

where:
R^{A} is H, CO₂H and its pharmaceutically acceptable salts, CO₂R^{C}, CONHR^{D}, CONR^{D}R^{E};
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl, (CH₂)ᵣ—C₃ to C₆ cycloalkyl;
each r is independently 0, 1, 2, 3, 4, 5 or 6;
said process comprising:
treating a first intermediate of the formula 6 with (1) an acylating reagent R^{B}COZ in which any hydroxyl group or groups in R^{B} are protected in the presence of a first base 8 and also in the presence of a first Lewis acid 9, (2) a palladium catalyst 10 with optional additional ligands 11 and (3) silica or an alternative equivalent solid reagent or a second mild base 12 followed by a Brønsted or second Lewis acid 13 or a mild base alone such as cesium acetate and optional deprotection to provide the second intermediate 7 and secondly hydrolysis of said 6 with optional decarboxylation or by transesterification or by amide formation with optional deprotection as appropriate to provide 5;

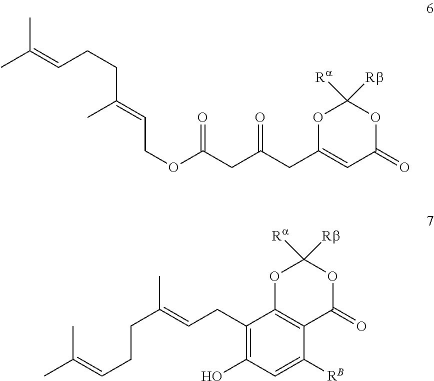

wherein:
Z is a halogen preferably chlorine or R^{B}COZ is an alternative reactive electrophilic acylating agent;
Rα and Rβ are independently C₁ to C₆ alkyl or optionally substituted aryl or Rα and Rβ in combination are (CH₂)ₛ (s is 4, 5 or 6) with Rα and Rβ being preferably both methyl;
the first base 8 is an amine or a heterocyclic amine such as pyridine;
the first Lewis acid 9 is preferably magnesium chloride;
the palladium catalyst 10 is either derived from a palladium(II) precatalyst or is itself a palladium(0) catalyst and the optional additional ligands 11 include but are not limited to one or more phosphines or diphosphines or their equivalents, preferably the palladium catalyst 10 and ligands 11 are specifically but not limited to phosphine complexes of palladium(0) such as tetrakis(triphenylphosphine)palladium(0) or tris(dibenzylideneacetone)dipalladium(0) [Pd₂(dba)₃] in the presence of a triarylphosphine or triheteroarylphosphine particularly tri-2-furylphosphine;
the second base 12 is cesium acetate or cesium carbonate or potassium carbonate;
the Brønsted or second Lewis acid 13, if used, is acetic acid or hydrogen chloride;
   wherein:
the optional hydroxyl-protecting group or groups are silyl protecting groups;
the optional hydroxyl-protecting group or groups are preferably independently t-butyldimethylsilyl, thexyldimethylsilyl, t-butyldiphenylsilyl or tri-iso-propylsilyl protecting groups.

It should be noted that several of the intermediates in these syntheses can exist as keto- and enol tautomers. The depiction of a structure as a keto-form also includes the corresponding enol-form including mixtures containing both keto- and enol forms. Additionally, the depiction of a structure as an enol-form also includes the corresponding keto-form including mixtures containing both keto- and enol forms. By way of example, intermediates 6 exist as mixtures of both keto- and enol forms although the structures, for reasons of simplicity, are drawn as the keto-forms.

The small-scale syntheses of intermediates 6 and 7 have previously been published (Rα and Rβ are both methyl; R^{B} is Me, AcOCH₂, trans-PhCH═CH) and are known [Ma, T. K.; White, A. J. P.; Barrett, A. G. M., Meroterpenoid Total Synthesis: Conversion of Geraniol and Farnesol into Amorphastilbol, Grifolin and Grifolic acid by Dioxinone-β-keto-Acylation, Palladium Catalyzed Decarboxylative Allylic Rearrangement and Aromatization, Tetrahedron Letters, 2017, 58, 2765-2767. Elliott, D. C.; Ma, T. K.; Selmani, A.; Cookson, R.; Parsons, P. J.; Barrett, A. G. M., Sequential Ketene Generation from Dioxane-4,6-dione-Keto-Dioxinones for the Synthesis of Terpenoid Resorcylates, Organic Letters 2016, 18, 1800-1803. Cordes, J.; Calo, F.; Anderson, K.; Pfaffeneder, T.; Laclef, S.; White, A. J. P.; Barrett, A. G. M., Total Syntheses of Angelicoin A, Hericenone J, and Hericenol A via Migratory Prenyl- and Geranylation-Aromatization Sequences, Journal of Organic Chemistry 2012, 77, 652-657]. However, methods for the large-scale synthesis of the novel cannabinoids 5 listed above have not been hitherto published.

Protecting groups are well known to persons skilled in the art and are described in textbooks such as Greene and Wuts, (P. G. M. Wuts, T. W. Greene, “Greene's Protective Groups in Organic Synthesis”, 2006, Fourth Edition, John Wiley, New York).

Cleavage of the dioxinone rings of intermediate 7 by saponification or an equivalent process to produce the cannabinoid carboxylic acids 5 (R^{A}═CO₂H) is carried out as described in R.

Cookson, T. N. Barrett and A. G. M. Barrett, “β-Keto-dioxinones and β,δ-Diketo-dioxinones in Biomimetic Resorcylate Total Synthesis”, Accounts of Chemical Research, 2015, volume 48, pages 628-642 and references therein.

Decarboxylation of the cannabinoid carboxylic acids 5 (R^{A}═CO₂H) is carried out as described in H. Perrotin-Brunel, W. Buijs, J. van Spronsen, M. J. E. van Roosmalen, C. J. Peters, R. Verpoorte and G.-J. Witkamp, “Decarboxylation of Δ⁹-tetrahydrocannabinol: Kinetics and molecular modeling”, Journal of Molecular Structure, 2011, volume 987, pages 67-73 and references therein.

Amide formation is carried out by activation of the carboxylic acid for example by formation of the N-hydroxysuccinimide ester and coupling with the corresponding amine, for example see Goto (Y. Goto, Y. Shima, S. Morimoto, Y. Shoyama, H. Murakami, A. Kusai and K. Nojima, “Determination of tetrahydrocannabinolic acid-carrier protein conjugate by matrix-assisted laser desorption/ionization mass spectrometry and antibody formation”, Organic Mass Spectrometry, 1994, volume 29, pages 668-671). Alternative amide coupling reagents include but are not limited to dicyclohexyl carbodiimide (DCC), di-iso-propyl carbodiimide (DIC), 0-(7-azabenzotriazol-1-yl)-1,1,3,3-tetramethyluronium hexafluorophosphate (HATU), 0-(benzotriazol-1-yl)-1,1,3,3-tetramethyluronium hexafluorophosphate (HBTU) and bromotri(pyrrolidino)phosphonium hexafluorophosphate (PyBrop) (E. Valeur and M. Bradley, “Amide bond formation: beyond the myth of coupling reagents”, Chemical Society Reviews, 2009, volume 38, pages 606-631).

The aforementioned novel cannabinoids with formulae 5 above may be used as active compounds either alone or admixed in combination with known cannabinoids such as but not limited to Δ⁹-tetrahydrocannabinol (THC), tetrahydrocannabivarin (THBV), cannabidiol (CBD) or cannabidivarin (CBDV) or other drugs for the treatment of pain, multiple sclerosis-related spasticity, nausea, epilepsy, Alzheimer's brain injury/concussion, cancer, infection, glaucoma and retinal degeneration, disorders of immune-inflammation, lung injury or disease, liver injury or disease, kidney injury or disease, eye injury or disease, amongst other pathologies. In some embodiments, the said novel cannabinoids with formulae 5 above either alone or admixed in combination with known cannabinoids such as but not limited to Δ⁹-tetrahydrocannabinol (THC), tetrahydrocannabivarin (THBV), cannabidiol (CBD) or cannabidivarin (CBDV) or other drugs are formulated into pharmaceutical compositions in a suitable form for administration to a patient. Such formulations, in addition to the active cannabinoid or cannabinoids in a combination therapeutic agent, contain pharmaceutically acceptable diluents and excipients, which may include binders such as lactose, starches, cellulose, sorbitol, polyethylene glycol or polyvinyl alcohol or other pharmaceutically acceptable oligosaccharides or polymers, disintegrants such as polyvinylpyrrolidone, carboxymethylcellulose or other pharmaceutically acceptable disintegrants, vehicles such as petrolatum, dimethyl sulfoxide, mineral oil, or in omega-3 oil-in-water nanoemulsions, or as complexes with cyclodextrins such as hydroxypropyl-beta-cyclodextrin, preservatives including antioxidants such as vitamin A, vitamin E, vitamin C, retinyl palmitate, cysteine, methionine, sodium citrate, citric acid, parabens or alternative pharmaceutically acceptable preservatives, antiadherents, lubricants and glidants such as magnesium stearate, stearic acid, talc, silica, pharmaceutically acceptable fats or oils, coatings such as cellulose ether hydroxypropyl methylcellulose, gelatin or other pharmaceutically acceptable coatings, flavors and fragrances such as but not limited to the volatile terpenes of Cannabis and citrus fruits and other pharmaceutically acceptable diluents or excipients. The aforementioned pharmaceutical compositions may be administrated to a patient by enteral administration for example as a pill, tablet or capsule, by sublingual administration for example as a tablet, strip, drops, spray, lozenge, effervescent tablet, intranasal administration for example as a spray or micronized powder, inhalation administration for example as a spray or micronized powder, rectal administration for example as a suppository or solution, by parenteral drug administration by intramuscular, subcutaneous or intravenous injection for example of a solution or by other known methods of clinical administration.

The aromatization reaction is suitable for the synthesis of novel cannabinoids 5 and these compounds are also part of the invention. The invention includes synthesis of the target cannabinoids as oils or crystalline derivatives, as appropriate, including solvates, hydrates and polymorphs. These novel cannabinoids 5 have the formula:

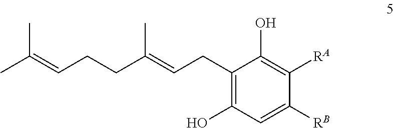

wherein:
R^{A} is H, CO₂H and its pharmaceutically acceptable salts, CO₂R^{C}, CONHR^{D}, CONR^{D}R^{E};
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl, (CH₂)ᵣ—C₃ to C₆ cycloalkyl;
each r is independently 0, 1, 2, 3, 4, 5 or 6;
   with the exception of cannabinoids cannabigerol (CBG, 1), cannabigerolic acid (CBGA, 2), cannabigerovarin (CBGV, 3) cannabigerovarinic acid (CBGVA, 4), and cannabinoids 5 [R^{A}═H with R^{B}═H, R^{B}═CH₃, R^{B}=n-C₃H₇, R^{B}═CH₂OH, R^{B}=n-C₅H₁₁, R^{B}=n-C₇H₁₅, R^{B}═CH₂OCH₃, R^{B}═CH₂CH₂ CH₂CH₂CH₂OH, R^{B}═C(CH₃)₂(CH₂)₅CH₃, R^{B}═CH₂(CHOH)-n-C₃H₇, R^{B}═C₂H₄(CHOH)-n-C₂H₅, R^{B}═C₃H₆(CHOH)CH₃], 5 [R^{A}═CO₂H with R^{B}=n-C₃H₇, R^{B}=n-C₅H₁₁], 5 [R^{A}═CO₂CH₃ with R^{B}═CH₃, R^{B}=n-C₃H₇, R^{B}=n-C₅H₁₁], and 5 [R^{A}═CO₂CH₂CH₃ with R^{B}=n-C₅H₁₁].

The dioxinone resorcylate derivatives 7 below, which are intermediates for the synthesis of cannabinoids, are also available by the synthetic routes herein described and are part of the invention. These novel dioxinone derivatives 7 have the formula:

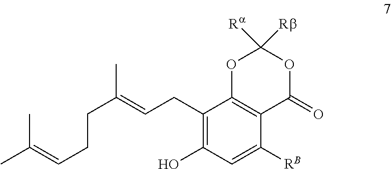

wherein:
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
o is 0, 1, 2, 3, 4, 5 or 6;
p is 1, 2, 3, 4, 5 or 6;
Rα and Rβ are independently C₁ to C₆ alkyl or optionally substituted aryl or Rα and Rβ in combination are (CH₂)ₛ;
s is 4, 5 or 6.
   with the exception of 7 (R^{B}=Me; Rα=Rβ=Me).

### EXAMPLES

### Example 1

### (E)-3,7-Dimethylocta-2,6-dien-1-yl 4-(2,2-dimethyl-4-oxo-4H-1,3-dioxin-6-yl)-3-oxobutanoate (6, R^{α}═R^{β}=Me)

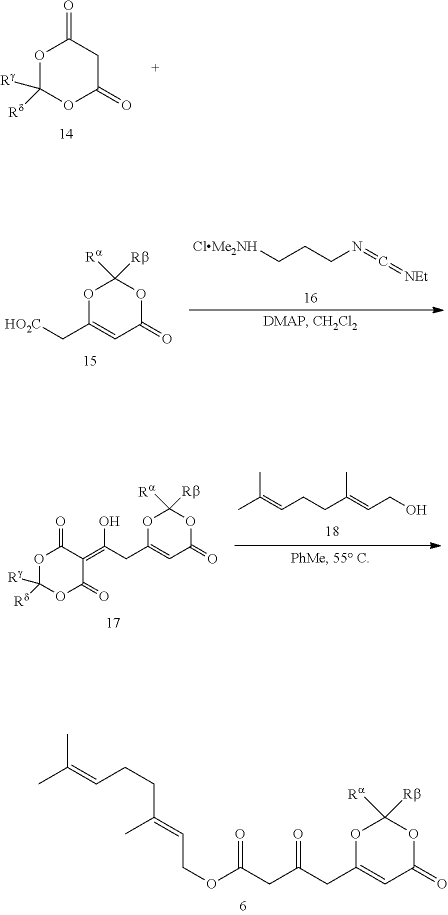

N-(3-Dimethylaminopropyl)-N′-ethyl carbodiimide hydrochloride (16) (2.6 g, 12.5 mmol) and 4-dimethylaminopyridine (DMAP) (1.5 g, 12.5 mmol) were sequentially added to a solution of 2-phenyl-1,3-dioxane-4,6-dione (14, R^{γ}=Ph, R^{δ}═H) (2.4 g, 12.5 mmol) in anhydrous dichloromethane (125 mL). After 5 minutes, 2-(2,2-dimethyl-4-oxo-4H-1,3-dioxin-6-yl)acetic acid (15, R^{α}═R^{β}=Me) (2.3 g, 12.5 mmol) was added with stirring in one portion. After 16 hours at room temperature, water (100 mL) was added and the organic fraction separated. The organic fraction was washed with 1M hydrochloric acid (2×100 mL) and brine (100 mL). The washed organic layer was dried over MgSO₄, filtered, and concentrated under reduced pressure. The crude product was immediately dissolved in anhydrous toluene (100 mL), and geraniol (18) (1.1 mL, 6.3 mmol) was added dropwise with stirring. The solution was heated to 55° C. and maintained at this temperature for 4 hours. Once the starting material had been consumed, the solution concentrated under reduced pressure. The crude reaction product was purified by flash column chromatography (EtOAc:pentane; 4:20), providing the title compound 6 (R^{α}═R^{β}=Me) as a colorless oil (1.9 g, 5.3 mmol, 84%): ¹H NMR (400 MHz, CDCl₃) δ 5.43-5.27 (m, 2H), 5.11-5.04 (m, 1H), 4.71-4.63 (m, 2H), 3.51 (s, 2H), 3.50 (d, J=0.5 Hz, 2H), 2.15-2.00 (m, 4H), 1.71 (s, 6H), 1.69 (s, 1H), 1.68 (d, J=1.3 Hz, 4H), 1.60 (d, J=1.4 Hz, 3H); ¹³C NMR (101 MHz, CDCl₃) δ 195.8, 166.5, 163.7, 143.8, 132.1, 123.7, 117.4, 107.5, 97.3, 62.8, 49.3, 47.1, 39.7, 26.4, 25.8, 25.2, 17.9, 16.7; IR (neat) 2966, 2917, 2856, 1718, 1636, 1388, 1270, 1200, 1014, 900 cm⁻¹; HRMS (ES+) m/z calculated for C₂₀H₂₉O₆[M+H]⁺ 365.1959, found 365.1968; Rf 0.14 (EtOAc:pentane; 4:20) UV/Vanillin.

### Example 2

### (E)-8-(3,7-Dimethylocta-2,6-dien-1-yl)-7-hydroxy-2,2-dimethyl-5-pentyl-4H-benzo[d][1,3]dioxin-4-one (7, R^{α}═R^{β}=Me, R^{B}=n-pentyl)

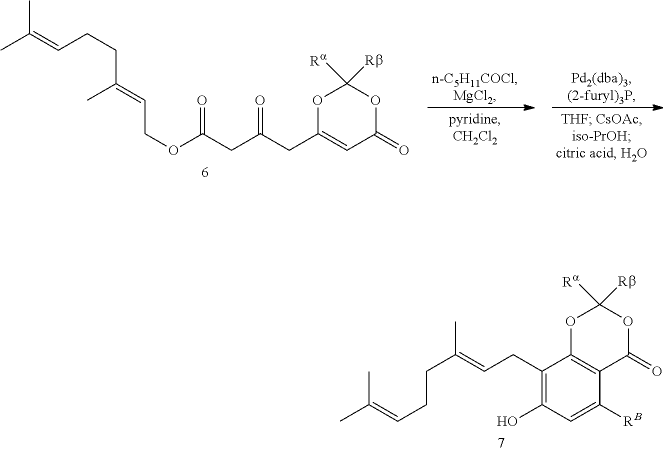

(E)-3,7-Dimethylocta-2,6-dien-1-yl 4-(2,2-dimethyl-4-oxo-4H-1,3-dioxin-6-yl)-3-oxobutanoate 6 (R^{α}═R^{β}=Me) (1.5 g, 4.1 mmol) was dissolved in dichloromethane (30 mL) cooled to 0° C. and pyridine (0.66 mL, 8.2 mmol) and MgCl₂ (0.4 g, 4.1 mmol) were added sequentially with stirring. After 5 minutes, n-hexanoyl chloride (R^{B}COZ, R^{B}=n-pentyl, Z=Cl) (0.75 g, 6.2 mmol) was added dropwise with stirring. After stirring for 1 hour at 0° C. and 2 hours at room temperature, saturated aqueous NH₄Cl (50 mL) was added and the biphasic mixture was subsequently acidified to pH 1 using 1M hydrochloric acid. The biphasic mixture was separated, and the aqueous partition was extracted with dichloromethane (2×50 mL). The combined organic fractions were washed with brine (100 mL), dried with MgSO₄, filtered, and concentrated under reduced pressure. The resultant oil was dissolved in THF (20 mL) and tri(2-furyl)phosphine (190 mg, 0.8 mmol) and tris(dibenzylideneacetone)dipalladium(0) (180 mg, 0.2 mmol) were added sequentially. After 1 hour, CsOAc in iso-propanol (0.5 M, 24 mL, 12 mmol) was added dropwise with stirring, and the reaction mixture was stirred for a further 1 hour. The reaction was quenched with 10% aqueous citric acid (100 mL), the biphasic solution was separated, and the aqueous layer was extracted with dichloromethane (3×40 mL). The organic extracts were combined and washed with brine (100 mL), dried over MgSO₄, filtered, and concentrated under reduced pressure. The residue was purified by flash column chromatography (dichloromethane:pentane; 1:1) to provide the title compound 7 (R^{α}═R^{β}=Me, R^{B}=n-pentyl) as a white solid (0.77 mg, 1.9 mmol, 47%): ¹H NMR (400 MHz, CDCl₃) δ 6.42 (s, 1H), 6.10 (s, 1H), 5.24-5.14 (m, 1H), 5.04 (dddd, J=7, 5.5, 3.5, 1.5 Hz, 1H), 3.32 (dd, J=7, 1 Hz, 2H), 3.04-2.94 (m, 2H), 2.16-1.99 (m, 5H), 1.79 (d, J=1.3 Hz, 3H), 1.67 (s, 7H), 1.75-1.52 (m, 6H), 1.66 (d, J=1.5 Hz, 3H), 1.58 (d, J=1.5 Hz, 3H), 1.34 (tq, J=5, 3 Hz, 5H), 0.93-0.82 (m, 3H); ¹³C NMR (101 MHz, CDCl₃) δ 160.6, 160.1, 156.1, 147.8, 138.6, 132.0, 123.7, 120.9, 112.9, 112.7, 105.0, 104.6, 39.7, 34.3, 31.9, 30.6, 26.4, 25.7, 22.6, 22.0, 17.7, 16.2, 14.1; IR (neat) 3291 (br), 2956, 2924, 2855, 1690, 1605, 1590, 1293, 1276, 1208, 1165, 1113, 1047 cm⁻¹; HRMS (ES+) m/z calculated for C₂₅H₃₇O₄[M+H]⁺ 401.2686, found 401.2686; Rf 0.28 (dichloromethane:pentane; 1:1) UV/Vanillin.

### Example 3

Cannabigerolic Acid (2)

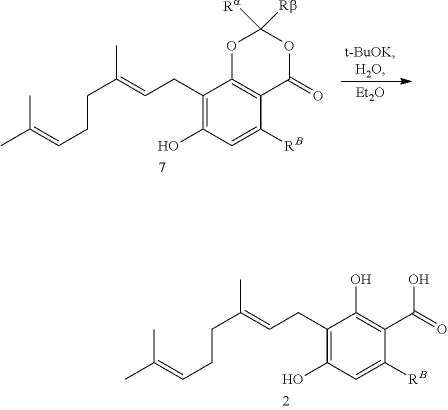

Potassium tert-butoxide (450 mg, 4 mmol) was suspended in Et₂O (5 mL) and (E)-8-(3,7-dimethylocta-2,6-dien-1-yl)-7-hydroxy-2,2-dimethyl-5-pentyl-4H-benzo[d][1,3]dioxin-4-one 7 (R^{α}═R^{β}=Me, R^{B}=n-pentyl) (200 mg, 0.5 mmol) and water (30 μL, 2 mmol) were added to the suspension. After 72 hours stirring, water (10 mL) and Et₂O (10 mL) were added and the biphasic mixture was phase separated. The organic layer was extracted with water (3×10 mL). The collected aqueous fraction was acidified with 4M hydrochloric acid (10 mL) until pH 1 was reached. The acidic solution was extracted with dichloromethane (3×10 mL) and the combined organic extracts were dried over MgSO4, filtered and concentrated under reduced pressure. The residue was purified by column chromatography (AcOH:EtOAc:pentane; 0.01:1:20) to give cannabigerolic acid (2) as a white powder (120 mg, 0.34 mmol, 68%): ¹H NMR (400 MHz, CD₃OD) a 6.20 (s, 1H), 5.21 (tq, J=7, 1.5 Hz, 1H), 5.05 (ddq, J=8.5, 6, 1.5 Hz, 1H), 3.27 (d, J=7 Hz, 2H), 2.91-2.76 (m, 2H), 2.09-2.00 (m, 2H), 1.95 (dd, J=8.5, 6.5 Hz, 2H), 1.76 (d, J=1.5 Hz, 3H), 1.59 (t, J=1.5 Hz, 4H), 1.58-1.48 (m, 4H), 1.41-1.27 (m, 4H), 0.96-0.87 (m, 3H); ¹³C NMR (101 MHz, CD₃OD) a 175.4, 164.7, 161.1, 146.8, 135, 132, 125.5, 124.2, 114.0, 110.9, 104.5, 40.9, 37.6, 33.2, 33.0, 27.7, 25.8, 23.6, 22.8, 17.7, 16.2, 14.4; IR (neat) 3534, 3400, 2959, 2911, 1635, 1610 1457, 1271, 1245, 1169, 754 cm⁻¹; H RMS (ES+) m/z calculated for C₂₂H₃₃O₄[M+H]⁺ 361.2373, found 361.2372; Rf 0.32 (AcOH:EtOAc:pentane; 0.01:1:20) UV/Vanillin.

### Example 4

Cannabigerol (1)

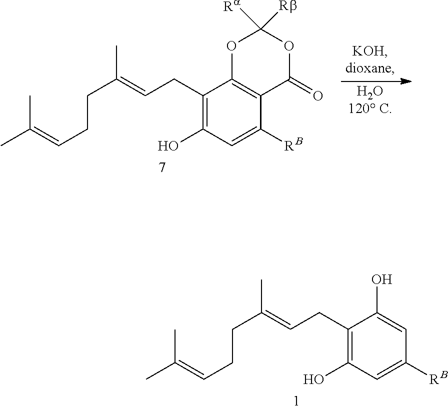

In a sealable reaction vial, (E)-8-(3,7-Dimethylocta-2,6-dien-1-yl)-7-hydroxy-2,2-dimethyl-5-pentyl-4H-benzo[d][1,3]dioxin-4-one 7 (R^{α}═R^{β}=Me, R^{B}=n-pentyl) (100 mg, 0.25 mmol) was dissolved in 1,4-dioxane (2.5 mL). Aqueous 5M KOH was (1.25 mL) was added and the biphasic mixture was sparged with nitrogen for 10 minutes. The reaction vial was sealed and heated to 120° C. for 18 hours. After cooling to room temperature, the reaction mixture was acidified with 4M hydrochloric acid (10 mL) with cooling, and the aqueous layer was extracted with EtOAc (3×20 mL). The combined organic extracts were washed with brine (20 mL), dried over MgSO₄, filtered, and concentrated under reduced pressure. The crude product was purified by column chromatography (Et₂O:pentane; 2:20) to provide cannabigerol (1) as a white powder (50 mg, 0.16 mmol, 64%): ¹H NMR (400 MHz, CDCl₃) δ 6.41 (s, 1H), 5.91 (s, 1H), 5.20 (tq, J=7.3, 1.3 Hz, 1H), 5.08-4.99 (m, 1H), 3.33 (d, J=7.2 Hz, 2H), 3.05-2.93 (m, 2H), 2.06 (tq, J=9.5, 5, 3.5 Hz, 4H), 1.79 (d, J=1.5 Hz, 3H), 1.67 (d, J=3 Hz, 9H), 1.59 (d, J=1.5 Hz, 6H), 1.45-1.24 (m, 5H), 0.94-0.82 (m, 5H); ¹³C NMR (101 MHz, CDCl₃) δ 160.6, 160.2, 156.2, 148.0, 139.0, 132.2, 123.8, 120.9, 113.0, 112.7, 110.1, 105.2, 104.7, 39.8, 34.4, 32.0, 30.8, 26.5, 25.9, 22.7, 22.1, 17.9, 16.4, 14.2; IR (neat) 3215, 2956, 2912, 2854, 1689, 1591, 1420, 1297, 912, 863 cm⁻¹; HRMS (ES+) m/z calculated for C₂₁H₃₂O₂[M+H]⁺ 316.2402, found 316.2402; Rf 0.22 (Et₂O:pentane; 2:20) UV/Vanillin.

1. A process for the preparation of a product compound of the formula 5: 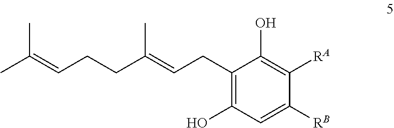
wherein:
R^{A} is selected from the group consisting of H, CO₂H and its pharmaceutically acceptable salts, CO₂R^{C}, CONHR^{D}, and CONR^{D}R^{E};
R^{B} is selected from the group consisting of H, C₁ to C₂ alkyl, linear or branched C₃ to C₁₀ alkyl, and double branched C₄ to C₁₀ alkyl, in each case optionally substituted by one or two hydroxyl groups or optionally substituted by one or more fluoro-groups, or is selected from the group consisting of (CH₂)ₒ—C₃ to C₆ cycloalkyl, (CH₂)ₚ—OR^{F}, and C₃ to C₆ cycloalkyl optionally substituted by a C₁ to C₈ alkyl;
o is an integer from 0-6;
p is an integer from 1-6;
R^{C} is selected from the group consisting of C₁ to C₆ alkyl, (CH₂)_{q}—C₃ to C₆ cycloalkyl, allyl, benzyl, substituted benzyl and 2-phenylethyl;
q is an integer from 0-6;
R^{D} is selected from the group consisting of C₁ to C₆ alkyl, (CH₂)ᵣ—C₃ to C₆ cycloalkyl, allyl, benzyl, substituted benzyl and 2-phenylethyl; and R^{E} is selected from the group consisting of C₁ to C₆ alkyl, (CH₂)ᵣ—C₃ to C₆ cycloalkyl, allyl, benzyl, substituted benzyl and 2-phenylethyl; or NR^{D}R^{E} is selected from the group consisting of azetidinyl, pyrrolidinyl, morpholinyl and piperidinyl, each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl or (CH₂)ᵣ—C₃ to C₆ cycloalkyl;
each r is an integer independently selected from 0-6;
Rα and Rβ are independently C₁ to C₆ alkyl or optionally substituted aryl, or Rα and Rβ in combination are (CH₂)ₛ, wherein s is 4, 5 or 6,
said process comprising the steps of:
providing a first intermediate of the formula 6: 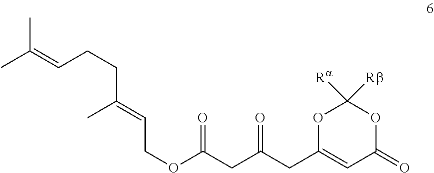
wherein Rα and Rβ are independently C₁ to C₆ alkyl or optionally substituted aryl, or Rα and Rβ in combination are (CH₂)ₛ, wherein s is 4, 5 or 6;
treating the first intermediate of the formula 6 with an electrophilic acylating reagent R^{B}COZ in which any hydroxyl group or groups in R¹ or R² is protected in the presence of a first base 8 and also in the presence of a first Lewis acid 9, a palladium catalyst 10 with optional additional ligands 11, and silica or an alternative equivalent solid reagent or a second base 12 followed by a Brønsted or second Lewis acid 13 or a base alone and optional deprotection to provide a second intermediate 7: 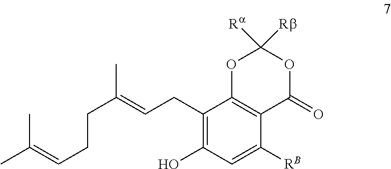
wherein:
Rα and Rβ are independently C₁ to C₆ alkyl or optionally substituted aryl, or Rα and Rβ in combination are (CH₂)ₛ, s is 4, 5 or 6; and
hydrolyzing the second intermediate 7 with optional decarboxylation or by transesterification or by amide formation with optional deprotection to provide the product of formula 5.

2. The process according to claim 1, wherein Z is a halide.

3. The process according to claim 1, wherein Rα and R^{β} are both methyl.

4. The process according to claim 1, wherein the first base 8 is an amine or heterocyclic amine.

5. The process according to claim 1, wherein the first base 8 is pyridine.

6. The process according to claim 1, wherein the first Lewis acid 9 is magnesium chloride.

7. The process according to claim 1, wherein the palladium catalyst 10 is derived from a palladium(II) complex in the presence of a phosphine 11 as ligand.

8. The process according to claim 1, wherein the palladium catalyst 10 is a palladium(0) complex in the presence of a phosphine 11 as ligand.

9. The process according to claim 1, wherein the palladium catalyst 10 is derived from a palladium(II) complex which contains one or more phosphine ligands.

10. The process according to claim 1, wherein the palladium catalyst 10 is a palladium(0) complex which contains one or more phosphine ligands.

11. The process according to claim 1, wherein the palladium catalyst 10 is tris(dibenzylideneacetone)dipalladium(0) [Pd₂(dba)₃] in the presence of a triarylphosphine or triheteroarylphosphine as ligand 11.

12. The process according to claim 1, wherein the second base 12 is cesium acetate, cesium carbonate or potassium carbonate.

13. The process according to claim 1, wherein the Brønsted or second Lewis acid 13, if used, is acetic acid or hydrogen chloride.

14. The process according to claim 1, wherein the hydroxyl-protecting group or groups are silyl protecting groups.

15. The process according to claim 1, wherein the hydroxyl-protecting group or groups are independently selected from the group consisting of t-butyldimethylsilyl, thexyldimethylsilyl, t-butyldiphenylsilyl or tri-iso-propylsilyl protecting groups.

16. A compound having the structure of formula 5: 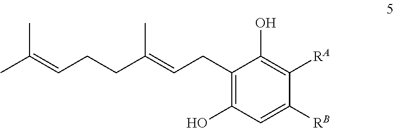
wherein:
R^{A} is selected from the group consisting of H, CO₂H and its pharmaceutically acceptable salts, CO₂R^{C}, CONHR^{D}, and CONR^{D}R^{E};
R^{B} is selected from the group consisting of H, C₁ to C₂ alkyl, linear or branched C₃ to C₁₀ alkyl, and double branched C₄ to C₁₀ alkyl in each case optionally substituted by one or two hydroxyl groups or optionally substituted by one or more fluoro-groups, or is selected from the group consisting of (CH₂)ₒ—C₃ to C₆ cycloalkyl, (CH₂)ₚ—OR^{F}, and C₃ to C₆ cycloalkyl optionally substituted by a C₁ to C₈ alkyl;
o is an integer from 0-6;
p is an integer from 1-6;
R^{C} is selected from the group consisting of C₁ to C₆ alkyl, (CH₂)_{q}—C₃ to C₆ cycloalkyl, allyl, benzyl, substituted benzyl and 2-phenylethyl;
q is an integer from 0-6;
R^{D} is selected from the group consisting of C₁ to C₆ alkyl, (CH₂)ᵣ—C₃ to C₆ cycloalkyl, C₃ to C₆ cycloalkyl, allyl, benzyl, substituted benzyl and 2-phenylethyl; and R^{E} is selected from the group consisting of C₁ to C₆ alkyl, (CH₂)ᵣ—C₃ to C₆ cycloalkyl, allyl, benzyl, substituted benzyl or 2-phenylethyl;
or NR^{D}R^{E} is azetidinyl, pyrrolidinyl, morpholinyl and piperidinyl, each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl or (CH₂)ᵣ—C₃ to C₆ cycloalkyl;
each r is an integer independently selected from 0-6;
with the exception that the compound of formula 5 cannot be cannabigerol (CBG, 1), cannabigerolic acid (CBGA, 2), cannabigerovarin (CBGV, 3) and cannabigerovarinic acid (CBGVA, 4).

17. An intermediate compound having the structure of formula 7: 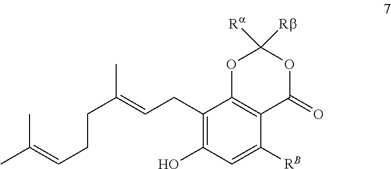
wherein:
R^{B} is selected from the group consisting of H or C₁ to C₂ alkyl, linear or branched C₃ to C₁₀ alkyl, and double branched C₄ to C₁₀ alkyl in each case optionally substituted by one or two hydroxyl groups or optionally substituted by one or more fluoro-groups, or is selected from the group consisting of (CH₂)ₒ—C₃ to C₆ cycloalkyl, (CH₂)ₚ—OR^{F}, and C₃ to C₆ cycloalkyl optionally substituted by a C₁ to C₈ alkyl;
o is an integer from 0-6;
p is an integer from 1-6;
Rα and Rβ are independently C₁ to C₆ alkyl or optionally substituted aryl, or Rα and Rβ in combination are (CH₂)ₛ, and is 4, 5 or 6,
with exception to when each of R^{B}, Rα and Rβ is Me.

18. The process according to claim 1, wherein the compound of formula 5 is cannabigerol (CBG, 1) or cannabigerolic acid (CBGA, 2).

19. The process according to claim 1, wherein the compound of formula 5 is cannabigerovarin (CBGV, 3) or cannabigerovarinic acid (CBGVA, 4).

20-23. (canceled)
